(19) United States
(12) Patent Application Publication
(10) Pub. No.: US 2015/0013568 A1
(43) Pub. Date: Jan. 15, 2015

(54) Glass Fillers Having Acid Resistance

(71) Applicant: Sukgyung AT Co., Ltd., Ansan-city (KR)

(72) Inventors: Hyung Sup Lim, Ansan-City (KR); Young Cheol Yoo, Ansan-city (KR); O Sung Kwon, Gunpo-city (KR); Jung Won Seo, Ansan-City (KR)

(21) Appl. No.: 14/330,446

(22) Filed: Jul. 14, 2014

Related U.S. Application Data
(60) Provisional application No. 61/846,256, filed on Jul. 15, 2013.

(51) Int. Cl. C08K 3/40; C08K 3/36; A61K 6/00

(52) U.S. Cl. CPC C08K 3/40; A61K 6/0091; C08K 3/36
USPC 106/35; 106/489; 106/450; 106/425; 106/483; 106/446; 106/482

(57) ABSTRACT

A glass filler manufacturing process and product which enable the manufacture of dental composite and dental cement products having superior product stability and also having superior physical properties. The process makes use of barium glass filler and strontium glass filler, which have high-radiopacity properties but could not previously be used for dental composites and dental cement. By simply coating the surface of the barium glass or strontium glass filler with an oxide having acid-resistance properties, and then following with a heat-treatment process, a suitable dental composite can be produced.

### RELATED APPLICATION

The present application claims the filing priority of U.S. Provisional Application No. 61/846,256, filed on Jul. 15, 2013. The '256 Provisional Application is hereby incorporated in its entirety by reference.

### TECHNICAL FIELD OF INVENTION

This present invention relates to glass fillers. Particularly, the invention relates to glass fillers with high mechanical strength and chemical durability.

### BACKGROUND OF THE INVENTION

Dental cement products are important dental treatment products and are typically hardened by an acid-base reaction. However, due to the properties of the product, the acids chiefly used for the acid-base reaction are organic acids having at least one double bond, such as acrylic acid or methacrylic acid.

The glass filler used is typically a silica glass filler, which has no radio-opacity properties. Contrastingly, the alternative barium glass filler and strontium glass filler have radio-opacity properties. The preference for silica glass filler is due to the stability of the product, which could not be assured if a barium glass filler or strontium glass filler is used. For example, when the alkali earth metal component, such as barium (Ba) and strontium (Sr), is dissolved, the hardening due to the dissolution causes hardening of the cement product itself.

To achieve the goal of product stability and in order to make a glass powder filler that is acid-resistant, alkali-resistant, and hydrolysis-resistant, Schott AG has commercialized a new product manufactured by melting materials with both high hydrolysis-resistance and acid-resistance (SiO₂ being the main component, with the quantities of alkali metals or alkaline earth metals being reduced, and acid-stable substances like ZrO₂ and La₂O₃ being added to produce glass) at high temperatures of at least 200-300° C. greater than used in manufacturing barium glass and strontium glass fillers of the prior art. However, because this process requires melting at very high temperatures, it has the drawbacks of (1) being difficult to ensure uniformity of the glass melt, and (2) having constrained mass-production due to the limitations of high-temperature furnaces. In addition, because it has a completely different composition from glasses currently in use, all evaluations of its use as dental filler must be done anew.

For at least these reasons, it will necessarily take considerable time before reliability of the Schott AG product can be assured. Accordingly, the Schott AG product has the further drawback that applications of the product will necessarily be limited.

Until the invention of the present application, these limitations and other problems in the prior art went either unnoticed or unsolved by those skilled in the art. After close observation and study of the foregoing product problems and with much experimentation, the current inventors have developed a product that addresses and solves each of these shortcomings of prior dental fillers. Specifically, a simple and inexpensive glass filler has been developed that, due to SiO₂ being coated evenly onto the surface of a barium glass filler and strontium glass filler used in the prior art, can maintain high radio-opacity properties while alleviating problematically poor acid-resistance.

### SUMMARY OF THE INVENTION

There is disclosed herein an improved glass filler which avoids the disadvantages and shortcomings of prior dental fillers while affording additional benefits and advantages.

An object of the disclosed process is to enable the manufacture of dental composite and dental cement products having superior product stability and also having superior physical properties. In a preferred embodiment, the process makes use of barium glass filler and strontium glass filler, which have high radio-opacity but could not previously be used for dental composites and dental cement. By simply coating the surface of the barium glass or strontium glass filler with an oxide having acid-resistance properties, and then following with a heat-treatment process, a suitable dental composite can be produced.

It is an object of the disclosed process to produce a dental composite product and dental cement product having superior stability and high radiopacity.

It is an object of the invention to provide an acid-resistant glass filler to the dental industry without using expensive elements, by imparting acid-resistance to barium glass filler and strontium glass filler currently used in dental products.

These and other aspects of the invention may be understood more readily from the following description and the appended drawings.

### BRIEF DESCRIPTION OF THE DRAWINGS

For the purpose of facilitating an understanding of the subject matter sought to be protected, there are illustrated in the accompanying drawings, embodiments thereof, from an inspection of which, when considered in connection with the following description, the subject matter sought to be protected, its construction and operation, and many of its advantages should be readily understood and appreciated.

FIG. 1 is a flow diagram illustrating a preferred manufacturing method for an embodiment of the disclosed glass filler;

FIG. 2 is a pair of photographs at high-power of dense glass powder particles coated with silicon dioxide after drying and heat treatment;

FIG. 3 is a graph showing alkali release test results using acetic acid (4% aqueous solution) for different glass powder products; and

FIG. 4 is a graph showing alkali release test results using acrylic acid (4% aqueous solution) for different glass powder products.

### DETAILED DESCRIPTION OF THE INVENTION

While this invention is susceptible of embodiments in many different forms, there is shown in the drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

Specifically, in the hardening tools based on acid/base reactions in dental composite and dental cement products, there has been no choice but to use a silica glass filler, which lacks radiopacity. The use of barium glass filler or strontium glass filler, which both have radiopacity, make it very difficult to ensure the stability of the dental composite or dental cement product. The product instability is a result of the hardening reaction taking place due to barium ions or strontium ions being produced by the elution of the barium component or strontium component of the product by an organic acid having at least one double bond, such as acrylic acid or methacrylic acid.

To alleviate this problem, the surface of the barium glass filler or strontium glass filler is coated with an oxide. It is then possible to affirmatively ensure stability of dental composite products and dental cement products because the elution of alkali metals and alkali earth metals by an organic acid, like acrylic acid or methacrylic acid, is then prevented.

Below is described the inventive process and the resulting inventive product filler. Specifically, the preferred process is described for producing a glass filler having superior acid-resistance, as is the product itself.

### Manufacturing Process

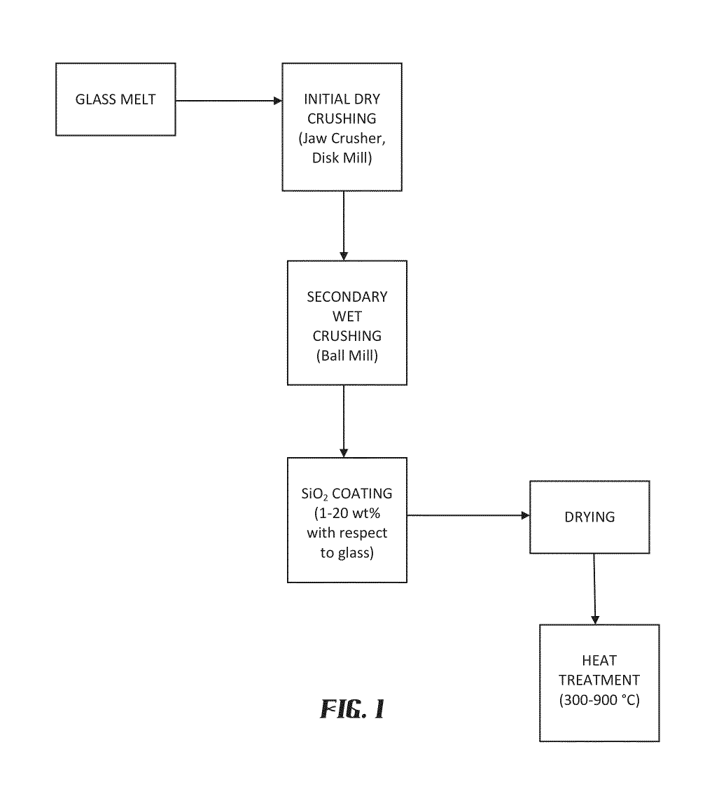

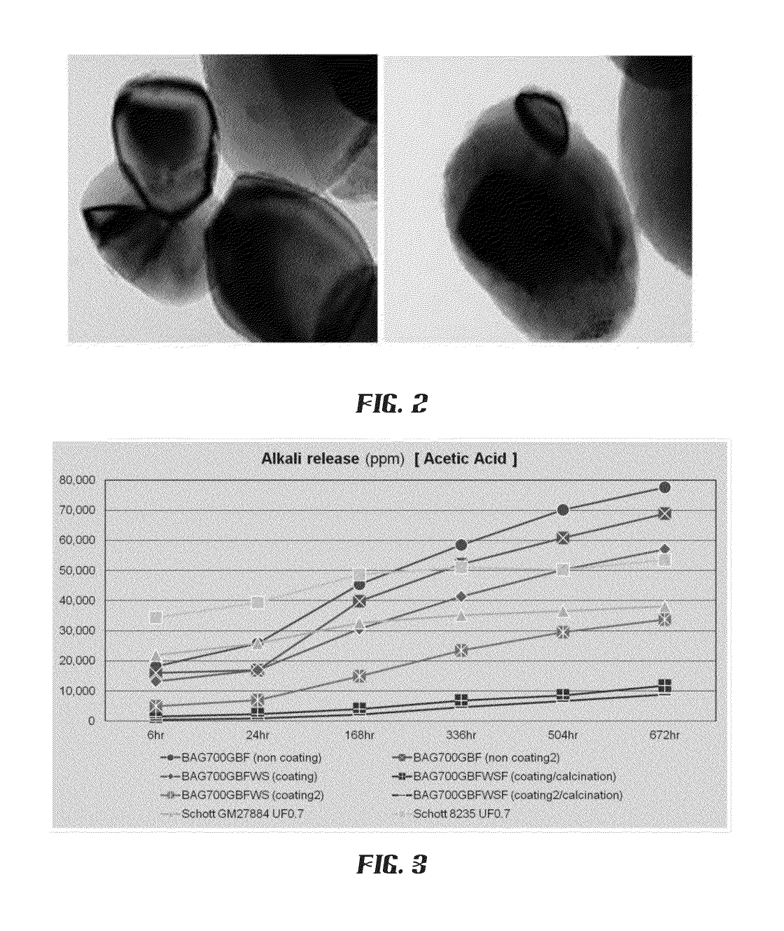

With reference to the flow chart of FIG. 1, the particles obtained from melting after mixing the raw materials in the original design composition are atomized to produce white micron-sized glass powder particles. This glass powder thus obtained is again made into a slurry at 10-50%. A silicon dioxide coating is then applied by means of a silicon (Si) source (1-20 wt % glass with respect to the quantity of SiO₂). Finally, as shown in the photos of FIG. 2, dense glass powder particles can be obtained by drying the silicon dioxide coated glass at a temperature in the range of 100-200° C., then heat treating the coated glass at a temperature in the range of about 300-900° C.

### Testing Product

Analysis of sample product made by the disclosed method found the glass powder to have a refractive index between 1.51-1.54. The preferred refractive index falls within the range of 1.4 to 1.6. In the case of a mixture of resins for dental composite use, an optical transmittance (ΔL*) in the range of 41-48 could be obtained.

To verify the acid resistance of the sample powder thus obtained, 1.0 gram of glass powder was placed in 20 mL of acetic acid (4% aqueous solution) and, after sonication for 5 minutes, the product was stored for 30 days at room temperature (about 18-20° C.), while an alkali release test was performed to analyze barium quantity using ICP-AES (model: ICPS-8100, manufacturer: SHIMADZU).

The results of the alkali release test are shown in TABLE 1 below and illustrated for comparison in the graph of FIG. 3.

TABLE 1

| | Alkali release (ppm) | | | | | | | |
|---|---|---|---|---|---|---|---|---|
| | | | | | | BAG700GBF | | |
| | | | | BAG700GBF | | WS | Schott | |
| | BAG700GBF | BAG700GBF | BAG700GBF | WSF (Coating/ | BAG700GBF | (Coating 2/ | GM27884 | Schott 8235 |
| Test period | (non coating) | (non coating 2) | WS (Coating) | calcination) | WS (Coating 2) | calcination) | UF0.7 | UF0.7 |
| 6 hr | 18,224 | 16,046 | 13,175 | 1,562 | 5,056 | 551 | 22,018 | 34,375 |
| 24 hr | 25,891 | 17,045 | 16,948 | 2,342 | 7,063 | 1,008 | 25,832 | 39,351 |
| 1 week (168 hr) | 45,373 | 39,837 | 30,726 | 4,009 | 14,891 | 2,170 | 32,705 | 48,698 |
| 2 week (336 hr) | 58,430 | 52,174 | 41,374 | 6,798 | 23,461 | 4,697 | 35,095 | 51,016 |
| 3 week (504 hr) | 70,162 | 60,791 | 50,185 | 8,558 | 29,573 | 6,736 | 36,593 | 50,256 |
| 4 week (672 hr) | 77,671 | 68,902 | 57,165 | 11,859 | 33,801 | 8,937 | 38,140 | 53,596 |

As a result, the product that was heat-treated at 700° C. following SiO₂ coating of the 700-nm Ba glass powder (i.e., coating with calcinations—sample nos. 4 and 6) had the lowest alkali elution at 9,000-12,000 ppm even after 720 hours, while the 700-nm Ba glass powder that was simply SiO₂-coated (i.e., sample nos. 3 and 5) was eluted at 30,000-60,000 ppm, and the uncoated 700-nm Ba glass powder (i.e., sample nos. 1 and 2) had a relatively high alkali elution of 70,000-80,000 ppm.

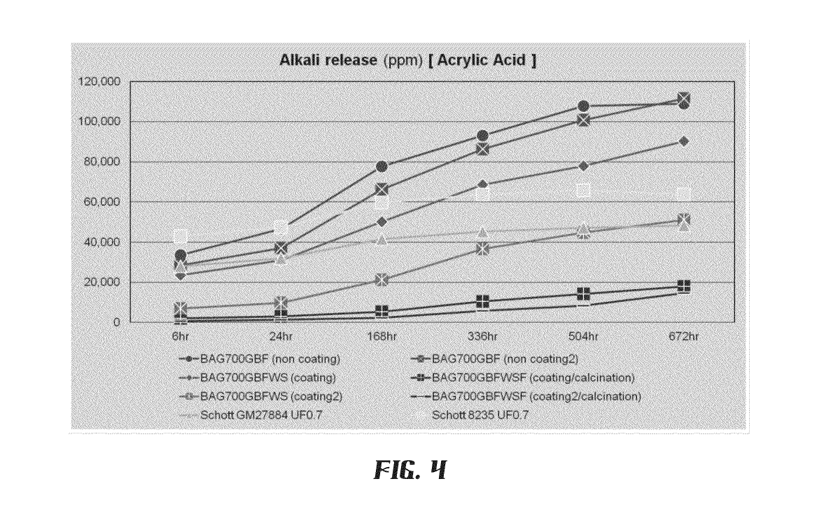

A second alkali release test was performed substituting acrylic acid (4% aqueous solution) for the acetic acid. The results of the second alkali release test are shown in TABLE 2 below and illustrated for comparison in the graph of FIG. 4.

TABLE 2

| | Alkali release (ppm) | | | | | | | |
|---|---|---|---|---|---|---|---|---|
| | | | | | | BAG700GBF | | |
| | | | | BAG700GBF | | WS | Schott | |
| | BAG700GBF | BAG700GBF | BAG700GBF | WSF (Coating/ | BAG700GBF | (Coating 2/ | GM27884 | Schott 8235 |
| Test period | (non coating) | (non coating 2) | WS (Coating) | calcination) | WS (Coating 2) | calcination) | UF0.7 | UF0.7 |
| 6 hr | 33,713 | 28,531 | 23,530 | 2,043 | 7,083 | 657 | 28,054 | 42,853 |
| 24 hr | 46,685 | 37,079 | 31,086 | 3,016 | 9,850 | 1,448 | 32,002 | 47,465 |
| 1 week (168 hr) | 77,553 | 56,256 | 50,180 | 5,445 | 21,346 | 2,436 | 41,650 | 59,680 |
| 2 week (336 hr) | 93,007 | 86,340 | 68,731 | 10,571 | 36,558 | 5,855 | 45,362 | 63,677 |
| 3 week (504 hr) | 107,908 | 100,760 | 77,983 | 14,277 | 44,822 | 8,439 | 47,142 | 85,867 |
| 4 week (672 hr) | 109,091 | 111,486 | 90,345 | 18,043 | 51,015 | 14,725 | 48,422 | 63,602 |

Clearly, results very similar to those of the first alkali release test were achieved with the 700-nm Ba glass powder having the lowest alkali elution.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A glass filler having acid-resistance performance, comprising:
a glass powder comprised of a plurality of particles and having radiopacity properties; and
a metal oxide forming a heat treated outer layer on the plurality of particles.

2. The glass filler of claim 1, wherein the metal oxide has a thickness in the range of about 1-10 nm.

3. The glass filler of claim 1, wherein the metal oxide coating comprises silicon dioxide (SiO2).

4. The glass filler of claim 1, wherein the metal oxide coating comprises a silicon dioxide composite selected from the group consisting of SiO₂—ZrO₂, SiO₂—ZnO, SiO₂—Al₂O₃, SiO₂—TiO₂, SiO₂—Yb₂O₃, SiO₂—La₂O₃, and SiO₂—Y₂O₃.

5. The glass filler of claim 3, wherein the glass filler has a refractive index in the range of 1.4-1.6.

6. The glass filler of claim 5, wherein the refractive index is in the range of 1.51-1.56.

7. The glass filler of claim 4, wherein the glass filler has a refractive index in the range of 1.4-1.6.

8. The glass filler of claim 7, wherein the refractive index is in the range of 1.51-1.56.

9. The glass filler of claim 1, wherein the filler is formulated as a dental composite.

10. The glass filler of claim 1, wherein the filler is formulated as a dental cement.

11. A method for forming an acid-resistant glass filler comprising the steps of:
melting a mixture of raw materials;
atomizing the melted mixture to produce micron-sized glass powder particles;
applying a metal oxide coating to the glass powder particles;
drying the coated glass powder particles; and
heat treating the coated glass powder particles.

12. The method of claim 11, wherein the step of drying the coated glass powder particles is done at a temperature in the range of about 100-200° C.

13. The method of claim 11, wherein the step of heat treating the coated glass powder particles is done at a temperature in the range of about 300-900° C.

14. The method of claim 11, wherein the metal oxide coating comprises silicon dioxide (SiO2).

15. The method of claim 11, wherein the metal oxide coating comprises a silicon dioxide composite selected from the group consisting of SiO₂—ZrO₂, SiO₂—ZnO, SiO₂—Al₂O₃, SiO₂—TiO₂, SiO₂—Yb₂O₃, SiO₂—La₂O₃, and SiO₂—Y₂O₃.

16. The method of claim 11, wherein the glass filler has a refractive index in the range of about 1.4 to about 1.6.

17. The method of claim 16, wherein the refractive index is in the range of about 1.51 to about 1.56.

18. The method of claim 11, wherein the raw materials have radiopacity properties.

19. The method of claim 11, wherein the metal oxide is applied to a thickness in the range of about 1-10 nm.

20. A glass filler having acid-resistance performance, comprising:
a glass powder comprised of a plurality of either barium components, strontium components or a mixture of barium and strontium components, the glass powder having radiopacity properties; and
a metal oxide forming an outer layer on the plurality of components, the metal oxide having a thickness in the range of about 1-10 nm, wherein the metal oxide is selected from the group consisting of SiO₂ or a SiO₂ composite.
